(19) United States
(12) Patent Application Publication
(10) Pub. No.: US 2014/0228599 A1
(43) Pub. Date: Aug. 14, 2014

(54) PRODUCTION OF ETHANOL FROM CELLULOSIC SUBSTRATES UTILIZING MICROORGANISMS

(71) Applicant: Dennis Chester, Santa Monica, CA (US)

(72) Inventor: Dennis Chester, Santa Monica, CA (US)

(21) Appl. No.: 14/179,049

(22) Filed: Feb. 12, 2014

Related U.S. Application Data
(60) Provisional application No. 61/764,757, filed on Feb. 14, 2013.

(51) Int. Cl. C12P 7/10

(52) U.S. Cl. CPC C12P 7/10
USPC 568/840; 435/165

(57) ABSTRACT

A continuous source of biofuel is disclosed that is formed from at least one colonizing entity, at least one cellulosic material and at least one carbohydrate conversion source. A process for producing a continuous source of biofuel comprises: providing at least one colonizing entity, providing at least one cellulosic material, providing at least one carbohydrate conversion source, combining the at least one colonizing entity, the at least one cellulosic material and the at least one carbohydrate conversion source to form a biofuel-precursor, incubating the biofuel-precursor at a temperature for a time period in order to produce a biofuel. In contemplated embodiments, the biofuel comprises ethanol.

This U.S. Utility Application claims priority to U.S. Provisional Application Ser. No.: 61/764757 filed on Feb. 14, 2013 and entitled “Production of Ethanol from Cellulosic Substrates Utilizing Microorganisms”, which is commonly-owned and incorporated in its entirety herein by reference.

### FIELD OF THE SUBJECT MATTER

The field of the subject matter is the production of ethanol from cellulosic substrates utilizing microorganisms, including mushrooms, fungi, yeast, bacteria and a combination thereof.

### BACKGROUND

Research into producing biofuels can be divided into several different areas, including producing and/or providing better starter materials, producing and/or providing better fermenters, improving the overall process to make the production of the biofuel more efficient, improving the overall process to make the production of the biofuel less expensive or a combination of some or all of the above.

Cellulosic (also may be referred to as lignocellulosic) substrates may be loosely defined as being made up of glucose or other sugars trapped within a matrix of bonds and other molecules. Consequently, the vast majority of animals cannot digest and produce energy from these widely divergent and available materials.

Cellulosic substrates are considered the “holy grail” in the production of alternate energy sources. One of those biofuels is ethanol. Today most bioethanol is obtained from food stocks, such as corn or sugar cane, and is considered non-optimal because the price of the food stock increases. Cellulosic substrates, on the other hand, can be various inexpensive grasses, weeds, and other items not considered food stock.

One of the key considerations that spans all of these areas is the desire to have a process that can efficiently operate on a continuous basis. In many instances, processes are still dependent on crop cycles and fermentation cycles, especially when specific crops are used for starter materials. Therefore, it would be ideal to review the fermentation process to determine what fermenters and related materials can operate on a continuous basis, and it would also be ideal to review starter materials to determine which of those are available and/or grow on a continuous basis without harming the land or removing a material that is otherwise considered a food source from agriculture, as opposed to the disclosure found in “Production of Bioethanol From Cassava and Sweet Potato Peels” by Oyeleke, S. B., et al., Advances in Environmental Biology, 6(1): 241-245.

The article “Characteristics of Wine Produced by Mushroom Fermentation” by Tokumitsu Okamura et al. (Biosci. Biotechnol. Biochem., 65 (7), 1596-1600, 2001) discloses a method of producing wine by using mushrooms. In this article, specific mushrooms were used as a substitute for Saccharomyces cerevisiae based on their ability to produce β-D-glucan in the final wine product, which the authors contend has some preventative effects against cancer and thrombosis. In this process, the mushrooms are harvested, cultured and the extract was used in the winemaking process, along with grapes. The process disclosed in this article is not continuous and requires the harvesting and cultivation of the mushrooms.

U.S. Patent Publication 2012/0149065 by Chris DaCunha et al. discloses a method of using manganese peroxidases, polynucleotides encoding improved manganese peroxidase and vectors and cells thereof, in the presence of a cellulose-containing biomass feedstock, to convert the feedstock to ethanol. This process uses a specific mushroom variety—the Trametes versicolor—to express the Mnp gene. There is no teaching or understanding of a universal process that can utilize any type of mushroom variety. There are many patent publications that utilize mushrooms as the starter materials, including 2006/0010714 and related applications, but clearly these starter materials are designed to be converted into a biomass and are not used in a continuous form.

One broad goal is to find a way to inexpensively and safely break up the cellulosic material found in these substrates to derive glucose and other sugars that yeast and other organisms can use as an energy source while producing ethanol and CO₂ as waste products. The ethanol may then be used as a readily available energy source. One may deduce that, if the cost to produce and distribute the bioethanol is significantly less expensive and safe, then a new industry would follow.

Therefore, it would be ideal to develop a process of producing ethanol that combines at least one of the following benefits: a) efficient source of ethanol, b) continuous source of ethanol, c) does not necessarily require a starter material that is a food source, d) utilizes the starter material as a substrate that will allow the production of a continuous culture, or a combination of all of the above. While many of the materials disclosed have been discussed in other publications, no one has been able to utilize the materials in a process similar to the ones disclosed herein.

### SUMMARY OF THE SUBJECT MATTER

A continuous source of biofuel is disclosed that is formed from at least one colonizing entity, at least one cellulosic material and at least one carbohydrate conversion source.

A process for producing a continuous source of biofuel comprises: providing at least one colonizing entity, providing at least one cellulosic material, providing at least one carbohydrate conversion source, combining the at least one colonizing entity, the at least one cellulosic material and the at least one carbohydrate conversion source to form a biofuel-precursor, incubating the biofuel-precursor at a temperature for a time period in order to produce a biofuel. In contemplated embodiments, the biofuel comprises ethanol.

### BRIEF DESCRIPTION OF THE FIGURE

FIG. 1 shows a contemplated embodiment, where a process includes: providing at least one colonizing entity 120, providing at least one cellulosic material 130, providing at least one carbohydrate conversion source 140, combining 150 the at least one colonizing entity, the at least one cellulosic material and the at least one carbohydrate conversion source to form a biofuel-precursor, incubating 160 the colonizing entity in the cellulosic precursor material and at least one carbohydrate conversion source to form the biofuel-precursor at a temperature for a time period in order to produce a biofuel. In contemplated embodiments, the biofuel comprises ethanol. In these embodiments, the phosphorus source, the nitrogen source or the combination thereof may be combined 155 with the at least one colonizing entity, the at least one cellulosic material and the at least one carbohydrate conversion source to form a biofuel-precursor. In some embodiments, the step or steps comprising incubating can be conducted under at least one environmentally optimum condition 165.

### DETAILED DESCRIPTION

According to contemplated embodiments, a process of producing ethanol has been disclosed that combines at least one of the following benefits: a) efficient source of ethanol, b) continuous source of ethanol, c) does not necessarily require a starter material that is a food source, d) utilizes the starter material as a substrate that will allow the production of a continuous culture, or a combination of all of the above. While many of the materials disclosed have been discussed in other publications, no one has been able to utilize the materials in a process similar to the ones disclosed herein.

A novel process has been developed around both fermentation materials and starter materials, wherein the combination of both the materials and the process provides an efficient and continuous source of ethanol without necessarily utilizing a food source starter material. That process, along with an example of how this process is utilized, will be disclosed herein. It should be understood, however, that the embodiments disclosed herein are designed to provide one of ordinary skill in the art of microbiology and or biofuel production information necessary to understand the process without being limited to the components disclosed herein. One of ordinary skill in the art of microbiology and or biofuel production—after reviewing this disclosure—would understand how many of the materials could be supplemented, augmented or otherwise substituted.

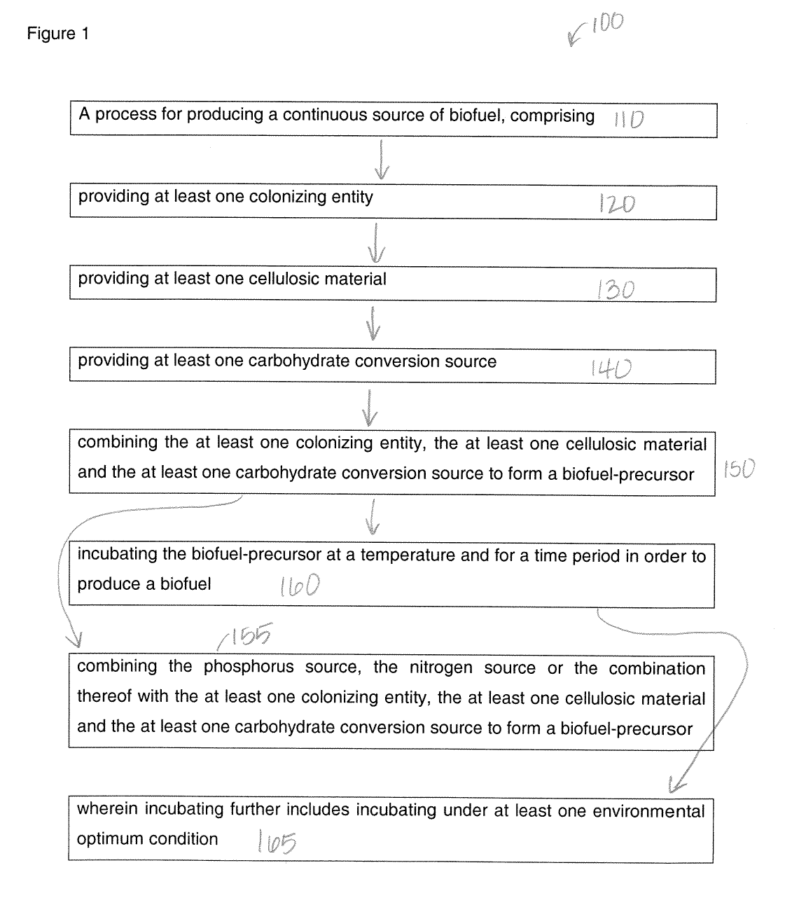

Specifically, a continuous source of biofuel is disclosed that is formed from at least one colonizing entity, at least one cellulosic material and at least one carbohydrate conversion source. A process 100 for producing 110 a continuous source of biofuel is shown in FIG. 1 and comprises: providing at least one colonizing entity 120, providing at least one cellulosic material 130, providing at least one carbohydrate conversion source 140, combining 150 the at least one colonizing entity, the at least one cellulosic material and the at least one carbohydrate conversion source to form a biofuel-precursor, incubating 160 the colonizing entity in the cellulosic precursor material and at least one carbohydrate conversion source to form the biofuel-precursor at a temperature for a time period in order to produce a biofuel. In contemplated embodiments, the biofuel comprises ethanol. In these embodiments, the phosphorus source, the nitrogen source or the combination thereof may be combined 155 with the at least one colonizing entity, the at least one cellulosic material and the at least one carbohydrate conversion source to form a biofuel-precursor. In some embodiments, the step or steps comprising incubating can be conducted under at least one environmentally optimum condition 165.

Mushroom mycelium is, for the purposes of this disclosure, considered equivalent to a plant and considered to be one embodiment of a colonizing entity. Whereas one-celled bacteria, as a rule do not coalesce, fungi do. When allowed to grow on a substrate, they will colonize and continue to live and metabolize just like a plant in a terrarium. They will thrive and continue to utilize new medium, just like a plant, and will continuously degrade cellulosic material on an ongoing basis (continuous culture), providing the carbon source necessary for yeast (carbohydrate conversion source) to produce alcohol.

Contemplated embodiments utilize several different components, including mushrooms and/or fungi (at least one colonizing entity), a yeast source (at least one carbohydrate conversion source) and at least one carbon substrate or cellulosic material (starter material).

It is contemplated that any suitable colonizing entity, mushroom, fungus or combination thereof can be utilized depending on the needs of the process, the carbon-based substrate and/or starter materials and the operating conditions. Contemplated mushrooms and/or fungi can be those bought in a typical grocery store, those purchased as stock cultures, those isolated from nature or a combination thereof. There are thousands of contemplated mushroom species, and contemplated embodiments may be augmented using the same protocol but utilizing additional mushroom species with varying degrees of success. In some embodiments, a contemplated colonizing entity comprises a fungus, an ascomycete (e.g. Penicillium), a bacterium or a combination thereof. In other embodiments, colonizing entities do not comprise an ascomycete. It should be understood for the purposes of this disclosure that ascomycetes is a Phylum within the group of fungi. In yet other embodiments, a contemplated colonizing entity comprises at least one mold.

A contemplated carbohydrate conversion source, such as a yeast, may come from any suitable supplier, including a winemaking supply store, a grocery store, or another suitable supplier, including being isolated by a natural source. The yeast used in the example below has been purchased from a wine making supply store. It should be understood that any suitable microorganism that can convert carbohydrates or sugars to carbon dioxide and alcohol may be utilized in the processes disclosed herein. In some embodiments, a contemplated carbohydrate conversion source comprises a yeast, a fungus, a bacterium or a combination thereof.

A contemplated carbon substrate, carbon-based substrate, organic substrate or cellulosic material, otherwise referred to as starter material, may comprise any suitable material, including waste materials from food, drink or agriculture processing, non-food based materials, and any suitable other material that can be transformed into a biofuel from the interaction of it with a contemplated colonizing entity and a contemplated carbohydrate conversion source. It should be understood that the at least one cellulosic material may be a food waste product or may not generally considered a food source. The phrase “food source” may mean a food source for humans, for animals, for insects or a combination thereof.

These materials, after inoculation, can be incubated for any suitable amount of time at any suitable temperature. It is understood that a suitable amount of time may include hours, days or weeks. It is also understood that suitable temperatures are greater than 10° C. In some embodiments, a suitable temperature is between 25 and 28° C. depending on the strain of organism and environmental conditions. In a practical sense, the time and temperature are selected in order to induce the enzymes to utilize the substrate and grow. Other conditions may be utilized to improve the methods contemplated herein, including pH variation, variation of mineral content, etc. In some embodiments, contemplated processes include providing at least one phosphorus source, at least one nitrogen source or a combination thereof. In these embodiments, the phosphorus source, the nitrogen source or the combination thereof may be combined with the at least one colonizing entity, the at least one cellulosic material and the at least one carbohydrate conversion source to form a biofuel-precursor.

In some embodiments, the step or steps comprising incubating can be conducted under at least one environmentally optimum condition. These “environmentally optimum conditions” include those conditions that are suitable for the materials, the location and the individual process, but they are not such that one of ordinary skill in the art can't model them or understand them within the bounds of biofuel production. Contemplated environmentally optimum conditions include increased humidity level, decreased humidity levels, atmospheric pressure, reduced pressure, increased pressure or a combination thereof. Contemplated environmentally optimum conditions further include moderation of oxygen level, pH level, degree of agitation or a combination thereof.

The example included herein is designed to provide several different pieces of information. First, the example shows a group of controls that should result in less or even no biofuel or ethanol production. These controls are comparative examples to be used in comparison with the contemplated embodiments. Second, this example shows a number of combinations of contemplated inventive embodiments. Third, this example shows a number of contemplated inventive conditions. As mentioned earlier, this example is ultimately designed to show both the grasp of these contemplated embodiments, along with the versatility and scope of contemplated embodiments.

### EXAMPLES

### Example 1

### Typical Contemplated Process

This example is designed to outline a typical contemplated process and comparative examples, along with providing some additional contemplated embodiments. In this example, it is understood that the process requires aseptic and sterile conditions unless stated otherwise.
1. Inoculate Pleurotus Ostreatus, Agaricus Bisporus, Lentinula edodes, and Saccharomyces cerevisiae on 5 plates of Potato Dextrose Agar with 3 inoculation points.
2. Incubate for 10 days at 27° C.
3. Inoculate each organism into ten 25 mL sterile disposable centrifugal tubes of Potato Dextrose Broth (PDB). Potato Dextrose Broth is the liquid form of PDA but is usually made per conventional formula found everywhere.
4. Pour 1 centrifuge tube of each into flasks of Rye, Straw, Manure, and BYI (Backyard infusion). These are selected in order to determine the ability of the strains of mushrooms to utilize the various substrates. Here they are each paired individually with four different substrates. For example, the oyster mushroom will have the chance to utilize rye, straw, horse manure, and BYI, which is basically dirt, grass, and plant clippings found in the back yard.
Flask 1=Pleurotus/Rye.
Flask 2=Pleurotus/Straw.
Flask 3=Pleurotus/Manure.
Flask 4=Pleurotus/BYI.
Flask 5=Agaricus/Rye.
Flask 6=Agaricus/Straw.
Flask 7=Agaricus/Manure.
Flask 8=Agaricus/BYI.
Flask 9=Shitake/Rye.
Flask 10=Shitake/Straw.
Flask 11=Shitake/Manure.
Flask 12=Shitake/BYI.
Flask 13=Yeast/Rye (control)
Flask 14=Yeast/Straw (control)
Flask 15=Yeast/Manure (control)
Flask 16=Yeast/BYI (control)
The substrates are all made up as slurries and are formulated close to published data and supplemented as needed. For instance, Rye Formulation is close except for the grinding of the rye prior to adding water and sterilizing. Note: All substrates are autoclaved.
5. Pour 1 centrifuge tube of each into a flask having a combination of Rye, Straw, and Manure to determine whether each mushroom will reproduce faster when the substrates are combined and whether each of the substrates will induce different enzymes (amylases, lactases, cellulases, etc.) that will allow for a better cleaving of cellulosic material freeing up reducing sugars for later use by yeast in producing ethanol.
Flask 17=Pleurotus/Rye, Straw, and Manure.
Flask 18=Agaricus/Rye, Straw, and Manure.
Flask 19=Shitake/Rye, Straw, and Manure.
Flask 20=Yeast/Rye, Straw, and Manure (control)
6. Pour 1 centrifuge tube of each into a flask having a combination of Rye, Straw, Manure and BYI.
Flask 21=Pleurotus/Rye, Straw, Manure, and BYI
Flask 22=Agaricus/Rye, Straw, Manure, and BYI
Flask 23=Shitake/Rye, Straw, Manure, and BYI
Flask 24=Yeast/Rye, Straw, Manure, and BYI.
7. Pour 1 centrifuge tube of Pleurotus, Agaricus, and Shitake (but no yeast) into a combination of Rye, Straw, and Manure to determine if the 3 different organisms can reproduce together when combined with all 3 organisms.
Flask 25=Pleurotus/Agaricus/Shitake/Rye, Straw, and Manure.
8. Pour 1 centrifuge tube of Pleurotus, Agaricus, and Shitake (but no yeast) into a combination of Rye, Straw, Manure and BYI.
Flask 26=Pleurotus/Agaricus/Shitake//Rye, Straw, Manure, and BYI.
9. Pour 1 centrifuge tube of Pleurotus, Agaricus, Shitake, and Yeast into a combination of Rye, Straw, and Manure in order to determine the role and the effect of the yeast.
Flask 27=Pleurotus/Agaricus/Shitake/Yeast/Rye, Straw, and Manure.
10. Pour 1 centrifuge tube of Pleurotus, Agaricus, Shitake, and Yeast into a combination of Rye, Straw, Manure, and BYI.
Flask 28=Pleurotus/Agaricus/Shitake/Yeast/Rye, Straw, Manure, and BYI.
11. Incubate each flask at 27° C.—Time Zero.
12. At 72 hours, shake each flask.
13. Shake each flask aerobically daily for 11 additional days while keeping culture sterile—use an air stone. Keep temperature at 27° C. The use of an air stone is similar to air stones used in aquariums and, in fact, an aquarium air pump is used. The pump is attached on one end to the culture solution and on the other a #2 Hepa Filter thus keeping the air sterile going into the flask.
14. Add 250 mL of Rye slurry mix into:
Flask 1.
Flask 5.
Flask 9.
Flask 13.
15. Add 250 mL of Straw slurry mix into:
Flask 2.
Flask 6.
Flask 10.
Flask 14.
16. Add 250 mL of Manure Broth into:
Flask 3.
Flask 7.
Flask 11.
Flask 15.
17. Add 250 mL of BYI Broth into:
Flask 4.
Flask 8.
Flask 12.
Flask 16
18. Add 80 mL of one or more substrate into:
Flask 17—80 mL each—Rye, Straw, and Manure Broth.
Flask 18—80 mL each—Rye, Straw, and Manure Broth.
Flask 19—80 mL each—Rye, Straw, and Manure Broth.
Flask 20—80 mL each—Rye, Straw, and Manure Broth.
19. Add 60 mL each of the following substrates into:
Flask 21—Rye, Straw, Manure, and BYI.
Flask 22—Rye, Straw, Manure, and BYI.
Flask 23—Rye, Straw, Manure, and BYI.
Flask 24—Rye, Straw, Manure, and BYI.
20. Add 80 mL each of the following substrates into:
Flask 25—Rye, Straw, and Manure.
21. Add 60 mL each of the following substrates into:
Flask 26—Rye, Straw, Manure, and BYI.
22. Add 80 mL each of the following substrates into:
Flask 27—Rye, Straw, and Manure.
23. Add 60 mL each of the following substrates into:
Flask 28—Rye, Straw, Manure, and BYI.
24. Remove 3 mL from each flask and test for ethanol and reducing sugars—first centrifuge—use supernatant. This is “time zero” for all combinations. There should be very little ethanol at this point. Reducing sugars should be tested and minimum at this time as well.
25. Incubate all flasks aerobically with shaking for 10 days at 27° C. Use air wand/filter once a day.
26. Take 3 mL from each flask and test for ethanol and reducing sugars—first centrifuge—use supernatant. Determine if the lactases, amylases, cellulases, etc. produce more reducing sugars. Are these enzymes still in solution? Was any alcohol produced by the mushrooms via an alternative pathway?
27. Pour off supernatant of each flask aseptically into identical labeled flasks with the prefix “AA.” The supernatant is expected to be turbid although somewhat clearer than when the media was introduced to the flasks.
28. Autoclave all flasks with prefix “AA.”
29. Inoculate yeast into all flasks with prefix “AA.”
30. Pour 100 mL of one or more of the following sterile Rye slurry, Straw slurry, Horse Manure slurry, and BYI slurry into:
Flask 29—Rye slurry—Yeast.
Flask 30—Straw slurry—Yeast.
Flask 31—Manure slurry—Yeast.
Flask 32—BYI slurry—Yeast.
Flask 33—Rye slurry—No Yeast.
Flask 34—Straw slurry—No Yeast.
Flask 35—Manure slurry—No Yeast.
Flask 36—BYI slurry—No Yeast.
Flask 37—Rye, Straw, Manure, and BYI slurries—No Yeast.
Flask 38—Rye, Straw, Manure, and BYI slurries—Yeast.
These are all controls and there should be no ethanol production from any of these controls.
31. Inoculate the following flasks with Yeast:
Flasks #AA—1-28.
Flasks 29-32.
Flasks 33-38.
32. Incubate all flasks labeled below at 27° C. with air lock and shaking:
Flasks #AA—1-28.
Flasks 29-32.
Flasks 33-38.
33. Test for Ethanol and reducing sugars Week 1, Week 2, Week 3, and continue until no more Ethanol is produced.
34. When there are no additional Ethanol production or sugars, pour off supernatant from each flask, centrifuge, and/or filter supernatant and refrigerate.
35. Add new substrate (slurry broth) to Flasks 1-28 as notated in #14-#23 and continue experiment for second generation. The second generation cuts down on additional incubation time allowing for more production of ethanol and is an initial step towards a continuous culture and production of ethanol.

### Example 2

### Contemplated Process with Analyticals

Analytical data is included from the analysis of five different flasks of contemplated mixtures used to produce biofuels.

Flask 3 substrate is horse manure, trace elements and assorted substances. For Flask 5 and Flask 9, the substrate is rye with trace elements and assorted substances. Each Flask contained a different mushroom species.

Flask 50 was a control for Flask 5 and Flask 58 was a control for Flask 3. Controls contained the exact substances and in the same amounts as their corresponding flasks but with no mushroom inoculation.

There was some alcohol produced in the control flasks, because some of the assorted substances contained sugars. For example, the rye berry does contain some sugars that can be metabolized by yeast to produce alcohol independently. However, the amount of alcohol is considerably less then the test flasks (especially the manure flask which does not contain rye). This result with the control flasks confirms the effectiveness of the contemplated embodiments represented in Flasks 3, 5 and 9.

Table 1 shows the ethanol production from each flask:

| | | | Reporting | | |
|---|---|---|---|---|---|
| Flask | Analyte | Result | Limit | Dilution | Method |
| 3 | Ethanol where the | 3400000 μg/L | 250000 | 5000 | EPA 8260B |
| | surrogate is | | | | |
| | dibromofluoromethane | | | | |
| | 90.4%, 86-118 | | | | |
| 5 | Ethanol where the | 32000000 μg/L | 250000 | 5000 | EPA 8260B |
| | surrogate is | | | | |
| | dibromofluoromethane | | | | |
| | 86.8%, 86-118 | | | | |
| 9 | Ethanol where the | 28000000 μg/L | 250000 | 5000 | EPA 8260B |
| | surrogate is | | | | |
| | dibromofluoromethane | | | | |
| | 89.8%, 86-118 | | | | |
| 50 | Ethanol where the | 17000000 μg/L | 250000 | 5000 | EPA 8260B |
| | surrogate is | | | | |
| | dibromofluoromethane | | | | |
| | 89.8%, 86-118 | | | | |
| 58 | Ethanol where the | 280000 μg/L | 250000 | 5000 | EPA 8260B |
| | surrogate is | | | | |
| | dibromofluoromethane | | | | |
| | 95.2%, 86-118 | | | | |

Table 2 shows the results for several analyses:

| | | | | | | | RPD | |
|---|---|---|---|---|---|---|---|---|
| | | | | | | | (Relative | |
| Batch | | | Reporting | Spike | | % REC | Percent | RPD |
| Identifier | Analyte | Result | Limit | Level | % REC | Limits | Difference) | Limit |
| B4B0560- | Ethanol where the | Analyte not | 50 | 50.0 | 115 | 86-118 | 0 | 0 |
| BLK 1 | surrogate is | detected | | | | | | |
| (Blank) | dibromofluoromethane | | | | | | | |
| | 57.4 | | | | | | | |
| LCS | Methyl tert-butyl ether | 53.3 μg/L | 0.50 | 50.0 | 107 | 80-120 | 0 | 0 |
| (B4B0560- | | | | | | | | |
| BS1) | | | | | | | | |
| LCS | Methyl tert-butyl ether | 52.6 μg/L | 0.50 | 50.0 | 105 | 80-120 | 0 | 0 |
| (B4B0560- | | | | | | | | |
| BS2) | | | | | | | | |
| LCS Dup | Methyl tert-butyl ether | 42.0 μg/L | 0.50 | 50.0 | 84 | 80-120 | 23.7 | 30 |
| (B4B0560- | | | | | | | | |
| BSD1) | | | | | | | | |

Thus, specific embodiments, methods of the production of ethanol from cellulosic substrates utilizing mushrooms and yeast have been disclosed. It should be apparent, however, to those skilled in the art that many more modifications besides those already described are possible without departing from the inventive concepts herein. The inventive subject matter, therefore, is not to be restricted except in the spirit of the disclosure herein. Moreover, in interpreting the specification, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

I claim:

1. A continuous source of biofuel that is formed from at least one colonizing entity, at least one cellulosic material and at least one carbohydrate conversion source.

2. The source of biofuel of claim 1, wherein the at least one colonizing entity comprises a fungus, an ascomycete, a bacterium or a combination thereof.

3. The source of biofuel of claim 1, wherein the at least one colonizing entity comprises a fungus, a bacterium or a combination thereof.

4. The source of biofuel of claim 1, wherein the at least one colonizing entity comprises at least one mold.

5. The source of biofuel of claim 1, wherein the at least one carbohydrate conversion source comprises yeast, fungus, or bacterium.

6. The source of biofuel of claim 1, wherein the at least one cellulosic material is not generally considered a food source.

7. The source of biofuel of claim 1, wherein the at least one cellulosic material comprises a food waste product.

8. A process for producing a continuous source of biofuel, comprising:
providing at least one colonizing entity,
providing at least one cellulosic material,
providing at least one carbohydrate conversion source,
combining the at least one colonizing entity, the at least one cellulosic material and the at least one carbohydrate conversion source to form a biofuel-precursor, incubating the biofuel-precursor at a temperature and for a time period in order to produce a biofuel.

9. The process of claim 8, wherein incubating further includes incubating under at least one environmental optimum condition.

10. The process of claim 9, wherein the at least one environmental optimum condition includes increased humidity level, decreased humidity level, atmospheric pressure, reduced pressure, increased pressure or a combination thereof.

11. The process of claim 9, wherein the at least one environmental optimum condition includes oxygen level, pH level, degree of agitation or a combination thereof.

12. The process of claim 8, wherein the process includes providing a phosphorus source, a nitrogen source or a combination thereof.

13. The process of claim 12, where the process includes combining the phosphorus source, the nitrogen source or the combination thereof with the at least one colonizing entity, the at least one cellulosic material and the at least one carbohydrate conversion source to form a biofuel-precursor.

14. The process of claim 8, wherein the at least one colonizing entity comprises a fungus, an ascomycete, a bacterium or a combination thereof.

15. The process of claim 8, wherein the at least one colonizing entity comprises a fungus, a bacterium or a combination thereof.

16. The process of claim 8, wherein the at least one colonizing entity comprises at least one mold.

17. The process of claim 8, wherein the at least one carbohydrate conversion source comprises yeast, fungus, or bacterium.

18. The process of claim 8, wherein the at least one cellulosic material is not considered a food source.

19. The process of claim 8, wherein the at least one cellulosic material comprises a food waste product.
